(19) United States
(12) Patent Application Publication
(10) Pub. No.: US 2022/0218634 A1
(43) Pub. Date: Jul. 14, 2022

(54) METHOD FOR INDUCING MASS APOPTOSIS OF CANCER CELLS BY ENHANCING THE OVERALL ACIDITY IN THE TUMOR

(71) Applicants: Chengde SHANG, Jieshou City (CN); Shengjie SHANG, Jieshou City (CN)

(72) Inventors: Chengde SHANG, Jieshou City (CN); Shengjie SHANG, Jieshou City (CN)

(21) Appl. No.: 17/214,897

(22) Filed: Mar. 28, 2021

(30) Foreign Application Priority Data
Jan. 13, 2021 (CN) 202110043906.1

(51) Int. Cl. A61K 31/19; A61K 9/00; A61P 35/00; A61F 7/00

(52) U.S. Cl. CPC A61K 31/19; A61F 7/00; A61P 35/00; A61K 9/0019

(57) ABSTRACT

A method for inducing mass apoptosis of cancer cells by enhancing the overall acidity in the tumor. The method enhances overall acidity in tumor internal tissue to reduce overall pH in tumor tissue, last to induce mass apoptosis of cancer cells. When pH of extracellular micro-environment of cancer cells is lower than 5.0, mass apoptosis of cancer cells can be induced. Base on the same direction synergic interaction between the changes of intracellular and extracellular acidity in tumor, the method may be achieved by injecting acid directly into tumor cells, or aggravating the anaerobic respiration of cancer cells through hyperthermia and other ways to enhance overall acidity in tumor tissues. The method may effectively induce mass apoptosis of cancer cells with little damaging to the normal cells, so as to inhibit the differentiation and proliferation of cancer cells, stop the metastasis and diffusion, and realize the radical cure of tumor.

### CROSS REFERENCE TO RELATED APPLICATION

This application claims the priority of Chinese Patent Application No. 202110043906.1, entitled “Method for inducing mass apoptosis of cancer cells by enhancing the overall acidity in the tumor” filed with China National Intellectual Property Administration on Jan. 13, 2021, which is incorporated herein by reference in its entirety.

### TECHNICAL FIELD

The disclosure relates to the medical field, and in particular to a method for inducing mass apoptosis of cancer cells by enhancing the overall acidity in the tumor.

### BACKGROUND ART

As we all know, surgical, chemotherapy, radiotherapy, targeted therapy, immunotherapy and gene therapy are the most general methods for the treatment of cancer. Most of these methods are similar to fishing with a net, only pay attention to treating big tumors, those small tumors become the small fish that escapes from the net, which is effective for early targeted tumors, but little effective for advanced tumor, especially the effect for invisible tiny tumors is even worse, so the spread tumors are hard to cure with current techniques.

After in-depth research, it is found that people are used to using modern technology to study the tumor problems that may be very simple originally, Due to artificially complicate the simple problems, which often leads the research to the wrong way thus often making the research fruitless, and as a result, the optimal chance to find a radical cure for cancer is lost. For example, in the research on “high fever cures cancer”cases, due to the lack of whole body concept of hyperthermia, undue emphasis is placed on the pursuit of focused thermal therapy for visible lumps, many hyperthermia techniques have been developed, ignoring the treatment of invisible tiny tumor of metastasis and diffusion, usually treat the symptoms, not the root cause, In the end leading to the failureon radical cure tumors, resulting the hyperthermia therapy technology with great potential and value in cancer treatment has not been fully recognized and effectively utilized. Another example, the essence of curing cancer by malaria and measles virus infection is also two good ways to cure cancer by using the principle of whole body hyperthermia. However, researchers unilaterally believe that curing cancer by malaria infection is due to the specific anti-cancer immunity of malaria and the function of killing cancer cells, and curing cancer by measles virus infection is due to the dissolve tumor enzyme function of measles virus. The researchers are used to using modern technology or esoteric theories to explain the mechanism that various viral infections may cure cancer. Precisely they all ignore the most basic fever characteristics of malaria and measles virus infection, and do not realize that these two kinds of virus infection cure cancers are both the effect of high fever. In clinical trials, instead in order to reduce and prevent the side effects of high fever, so much so that they deliberately control fever, inadvertently cover up the effect of high fever, reducing the effect of treatment, failing to find out the mechanism of treatment. As a result, the opportunity to find a radical cure for cancer was missed time and time again.

### SUMMARY OF THE INVENTION

In order to solve the problem that the tumor is difficult to cure, the present disclosure provides a method for inducing the mass apoptosis of cancer cells by enhancing the overall acidity in the tumor, based on the causal relationship between the acidity change in the tumor and the cancer apoptosis.

The present disclosure provides the following technical scheme.

A method for inducing mass apoptosis of cancer cells by enhancing overall acidity in tumor, wherein the method comprises steps of reducing overall pH in tumor tissue by adopting a physical or biochemical method, so as to enhance overall acidity in tumor tissue and induce mass apoptosis of cancer cells.

In one embodiment, the step of reducing the overall pH in tumor tissue includes needing to make the pH of extracellular micro-environment of cancer cells in tumor lower than 5.

In one embodiment, for reducing overall pH in tumor tissue may be performed by injecting an acid directly into tumor tissue or by aggravating anaerobic respiration of cancer cells through hyperthermia therapy to indirectly promote the overall acidification in tumor tissue, base on there are the same direction synergic interaction between the changes of intracellular acid-base property and extracellular acid-base property of cancer cells in tumor.

In one embodiment, for a single tumor without metastasis and diffusion, a liquid of an acid substance is injected directly into tumor tissue to promote the pH of extracellular micro-environment of cancer cells to be lower than 5, thus inducing mass apoptosis of cancer cells.

In one embodiment, for a single tumor without metastasis and diffusion, the hyperthermia therapy can be applied to the tumor tissue region to aggravate the anaerobic respiration of cancer cells and enhance acidity of cancer cells intracellular tissues and of cancer cells extracellular tissues, intent is to promote the pH of extracellular micro-environment of cancer cells to be lower than 5, thus fully enhancing the overall acidity in tumor and inducing mass apoptosis of cancer cells.

In one embodiment, for metastatic and diffuse tumors, the hyperthermia therapy may be applied to the patient whole body to aggravate the anaerobic respiration of cancer cells, and enhance acidity of the cancer cells intracellularly tissues and of the cancer cells extracellular tissues, intent is to promote the pH of extracellular micro-environment of cancer cells to be lower than 5, thus fully enhancing the overall acidity in tumors and inducing mass apoptosis of cancer cells.

In one embodiment, the internal temperature of tumor tissue is 43° C.-44° C. during the hyperthermia therapy.

In one embodiment, during the hyperthermia therapy, the optimal temperature range for the hyperthermia therapy is 43° C.-44° C., the hyperthermia therapy is carried out once a day, with an accumulated time for each hyperthermia therapy being not less than 30 minutes, in order to intensive effect the hyperthermia therapy is repeated once the next day.

Return to the origin of “high fever cures cancer” to explore its therapeutic mechanisms, We found that the root cause of the poor curative effect of hyperthermia therapy technologies of microwave and radiofrequency is: first of all, many cutting-edge thermal therapy technologies due to the lack of whole body heated concept, the hyperthermia therapy only focus on heating large tumor mass, and ignore the curing of small cancer cells, “treat the symptoms, not the root cause”, therapy must be failure. Secondly, it is limited by technology, there is a lack of accurate thermometry technology Inside body. In order to the prevention and control of high heat risk, equipment manufacturers do not take a risk to set the temperature to an optimal critical high temperature, which greatly reduces the effect of thermal therapy. After making certain the defects of the existing thermal therapy technology, useing the simplest thinking to analyze the most complex problems may see through the appearance to perceive its essence. Further research shows that the essence of high fever cureing cancer is not that hyperthermia therapy burns cancer cells to death, but that hyperthermia therapy causes the rapid enhancing of intracellular acidity in cancer cells. The enhancing of intracellular acidity in cancer cells can start the apoptosis program of cancer cells and induces the apoptosis of cancer cells, which makes cancer cells lose the function of differentiation and proliferation. Therefore, the enhancing of intracellular acidity in cancer cells can induce apoptosis of cancer cells, which is the root and the mechanism of high fever curing cancer.

In order to further research why Hot acidification can lead to the cancer cells apoptosis, we must carefully research the characteristics of tumor tissue structure and biological evolution, therefrom find out the reasons why cancer cells proliferate indefinitely and grow wildly out of control and never die, and then we find out their defects in order to realize technologies breakthrough. First of all, we should seek a breakthrough from the difference between apoptosis of cancer cells and necrosis of cancer cells. The trees in autumn are suddenly attacked by cold current and cold wind, and the leaves will quickly turn yellow and fall. The cold wind is the inducement of the leaves to fall. After the leaves are stimulated by the cold wind, their nutrition will flow back to the trunk, those leaves are more quickly wither and fall off. The sacrifice of the leaves lets the trees to survive better, to promote trees own dormancy, to reduce respiratory consumption, and to better overwinter. This is a typical phenomenon of biological natural apoptosis, this kind of apoptosis is conducive to the survival of species. It may be seen that cell apoptosis refers to the slow death program of cell caused by the pre-existing death program triggered by internal and external factors. It is spontaneous and orderly apoptosis of cells controlled by genes for self-protection under the action of radiation, high heat, acidity and virus. Cell necrosis is an early reaction of cells when they are injured by acute forces. First, the cell membrane is directly destroyed, a large amount of moisture enters the cells, the nuclear chromatin is flocculent, protein synthesis slows down, then the cytoskeleton is destroyed, the lysosome is disintegrated, and finally the cell membrane and organelle are broken, and the intracellular content flows out. The biggest feature is that it will cause inflammation in the surrounding tissues. By comparing shows obviously different between apoptosis and necrosis. Apoptosis is a active death and necrosis is a passive death. Moreover, because apoptosis is always sealed by membrane and no content is released, they will not cause inflammation, and will eventually be phagocytosed and digested by neighboring cells, which greatly reduces the harm to the organism itself. Based on the difference between apoptosis and necrosis of cancer cells, it is a good research direction to find a method that only allows tumor apoptosis without necrosis and also does not harm normal cells in order to conquer the tumor problems. Researches of us have found that cancer cells are very fragile and extremely vulnerable to high fever, and the high fever above 44° C. may cause cancer cell necrosis, and secondary inflammation of necrosis will harm normal tissues, which has no research value, but high fever of temperature higher than 39° C. not higher than 44° C. may induce mass apoptosis of cancer cells, and will not cause cancer cell necrosis and body inflammation, it will not also cause great harm to normal tissue cells. By mass apoptosis of cancer cells to stop differentiation and to reduce the consumption of proliferation is a beneficial result of the cancer cells maximizing self-protection biological characteristics, To be able to make cancer cells no longer differentiate and proliferate, In effect it's equal to can actually cure cancer, So it is of great significance to research the morphological change in mass apoptosis of cancer cells.

In-depth research found that the formation mechanism of tumor thermal acidification apoptosis is related to three prominent characteristics of cancer cells:

(1) The differentiation characteristics of cancer cells. First, normal cells all generally age and undergo apoptosis, while the apoptosis program of cancer cells is locked under normal circumstance, the death is inhibited, and they will not undergo apoptosis. As a result, they only increase without decreasing. Cancer cells are called “immortal cells”; Secondly, cancer cells differentiates fast and proliferates eternally with a loss of control of proliferation. The body's immunity is insufficient, and is ineffective to kill and inhibit the cancer cells, therefore the cancer cells are easy to metastasize and diffused; Thirdly, the metabolism of cancer cells is extremely strong, and the oxygen consumption and nutritional requirements are much higher than those of normal cells.

(2) The vascular characteristics of cancer cells. First, the blood vessels in the tumors are insufficient, and the vessel wall is easy to rupture; secondly, the structure of the blood vessels is abnormal and the tissue is particularly disordered; thirdly, the blood vessels are twisted and deformed, and the microvessels are elongated; fourthly, the vascular sinus is wide and there are more arteriovenous fistulas; fifthly, the blood vessels of cancer cells are easy to embolize and the blood flow resistance is large. Cancer cell differentiation is limited by the above-mentioned vascular defects, it still proliferates quickly when its logistics supply is obviously insufficient, otherwise the proliferation will be even more crazy.

(3) The being heated characteristics of cancer cells. Under normal circumstances, cancer cells need to rely on anaerobic respiration to achieve rapid proliferation under the condition of insufficient blood vessel oxygen supply, which will cause the accumulation of lactic acid, leading to a tendency of tumors continuously acidification. In order to avoid the influence of intracellular acidity to their own survival, the cancer cells have evolved the ability to autonomously reduce their intracellular acidity: the cancer cells are capable of using limited tumor vascular channels and carrier proteins for maximize the delivery of oxygen and nutrients inward, at the same time continuously transporting H⁺ from the inside of the cell to the outside, in order to reduce intracellular acidity. It is currently known that in tumor tissues under normal circumstances, the intracellular of cancer cells show weakly alkaline but not acidic (pHi7.1-7.6), and only the extracellular fluid of cancer cells is acidified (pHe6.2-7.1), the acid-base characteristic of the intratumoral tissue (intracellular weak alkalinity of cancer cells and extracellular acidification of cancer cells) are very suitable for cancers survival, differentiation and proliferation, and can not activate the apoptosis program of cancer cells. However, when the tumor is heated, the respiration of cancer cells is accelerated. Due to the congenital defects of the tumor blood vessels, the internal hypoxia of the cancer cells becomes more serious. Stress anaerobic respiration inside the cancer cells intensifies, then cancer cells produce more lactic acid instantly. Although cancer cells have the ability to transport H⁺ from the inside of the cells to the outside, but when the cancer cells are heated, the anaerobic respiration produces too much lactic acid, and the cancer cells don't have enough time to move all of the acid out of the cells. the result lead to accumulate acidity in intracellular and to aggravate acidity in extracellular of the cancer cells, here can be found that the changes between intracellular acidity and extracellular of cancer cells have the same direction synergic interaction, the accumulation of intracellular lactic acid in cancer cells leads to aggravate extracellular acid, finally, the overall acidification of intracellular and extracellular cancer cells in the tumor is caused, the overall acidification of the tumor signals that the logistics supply is obviously insufficient. When the enhancement of tumor acidity reaches a specific critical point, the inherent protection program of cancer cells will be automatically triggered, and the locked apoptosis program will be activated again stopping protein synthesis and DNA replication so as to reduce the consumption of cancer cell proliferation and achieve self-protection of the cancer cell group, resulting Tumor cells lose their ability to differentiate and proliferate. Afterwards, these apoptotic cancer cells gradually become senescent, then they are recognized and phagocytosed by immune cells. It can be seen that the enhancement of the overall acidity in the tumor (intracellular and extracellular of cancer cells) is the internal reason for inducing cancer cell apoptosis, and it is also the medical mechanism of the cases of “high fever can cure cancers”, and it also finds a way to cure the tumors.

It is found in the research that the acid-base characteristics of tumor tissues (intracellular weak alkalinity and extracellular acidification in cancer cells) are relatively stable under normal circumstances, and the acid-base characteristics of tumor tissues are generally difficult to change. It is difficult to change the overall acid-base characteristics of the tumor by conventional methods such as intravenous injection. Therefore, it is a very important topic with practical significance as to how to induce mass apoptosis of cancer cell by enhancing the overall acidity in the tumor.

Through repeated experiments and analysis, two ways to induce tumor cells mass apoptosis through enhance the overall acidity of the tumor are found: one is, as mentioned above, hyperthermia therapy may promote acidification of tumor through aggravating the anaerobic respiration in cancer cells, since there is no enough time for all the H⁺ to be transported from the inside of the cancer cell to the outside during hyperthermia therapy, and because the changes between intracellular acidity and extracellular of cancer cells have the same direction synergic interaction , so the tumor tissue is overall acidulated in the end. It is found that heating the tumor at a temperature higher than 39° C. can lead to overall acidification of the tumor tissue, at this time the pH of the extracellular micro-environment of cancer cells in the tumor is lower than 6.2, the cancer cells automatically initiate the apoptosis program and the tumor begins to slow down the rate of differentiation and proliferation. When the hyperthermia temperature exceeds 42° C. capproaches the temperature limit 44° C., the actual measurement shows that the extracellular acidity of cancer cells in the tumor rapidly increases to pH<5, the apoptosis rate of cancer cells increases rapidly and approaches 100%, which actually induce the mass apoptosis of cancer cells. At this temperature, the blood vessels of normal tissues have not obvious defects due to long-term evolution, and the increase of apoptosis is not obvious in normal tissue , and hyperthermia does little damage to normal tissue. The second is to directly inject acidic agents into the tumor. When the injected acid makes the pH of the extracellular micro-environment of cancer cells in the tumor being lower than 6.2, the cancer cells can also initiate the apoptosis program. The lower the pH of the extracellular micro-environment of cancer cells in the tumor is, the higher the apoptosis rate of cancer cells is. When the acidity of the extracellular micro-environment of cancer cells in the tumor increases to pH<5 for a sufficient period of time, the apoptosis rate of cancer cells can eventually approach 100%, i.e. it can also induce mass apoptosis of cancer cell, and then the tumor stops differentiation and proliferation of cells, and finally gradually atrophies and dies. Through research and analysis, it is found that directly injecting an acid into the tumor through human intervention can enhance the acidity of extracellular tissues of cancer cells in the tumor, which can inhibit the transport of acidic substances in cancer cells to the outside of the cancer cells, resulting in the enhancement of intracellular acidity in cancer cells, in fact which enhance the overall acidity of tumor tissue and induce apoptosis of cancer cells. The researches also foud that enhancing the overall acidity of tumor internal tissues is a very limited inducement in promoting cancer cells apoptosis. It is of great significance to find the biological characteristics that enhancing the overall acidity in the tumor can induce cancer cells apoptosis. This discovery not only may provide a basis for overcoming the problem of tumors, but also can form the latest theory that “mass apoptosis of tumor cells can be induced by enhancing the overall acidity in the tumor”.

It is also found that the changes between intracellular acidity and extracellular of cancer cells have the same direction synergic interaction when the acidity inside the tumor increased to pH<6.5, whether to enhance the cancer cell acidity from the inside or from the outside, In the end the acid-base property of the extracellular and the intracellular of the cancer cell is the same. this means that the acidity of the whole tumor tissue (intracellular and extracellular of cancer cells) can be increased regardless of whether the acidity is enhanced from the outside to the inside or from the inside to the outside. This discovery avails to avoid to determine directly the intracellular acidity of cancer cells,becaue it is very difficult to determine directly the intracellular acidity of cancer cells. On the contrary, with this finding it can be very convenient to study the law of intracellular acidification and apoptosis of cancer cells merely based on the changes of extracellular acidity of cancer cells.

At present, there are few researches on cancer apoptosis by direct acidification or by indirect acidification. A few similar studies have not obtained theoretical breakthroughs due to insufficient research depth. First, not yet researches have found that there is a minimum acidity criticality requirement for enhancing overall acidity in tumor to induce cancer cell apoptosis. Generally researches are conducted in the range of pH>6.2, and not yet researches have found that the increase of acidity to pH<5 has a causal relationship with the mass apoptosis of cancer cells; second, not yet researches have found that tumor heated may aggravate anaerobic respiration, which leads to the enhancement of overall acidity in tumor, and then induces mass apoptosis of cancer cells. Not yet researches have found that there are also strict requirements for the critical temperature of the hyperthermia therapy, we find this critical temperature range is about 43° C. at the lowest and 44° C. at the highest. The traditional thermal therapy mostly focuses on study of the temperature range below 43° C., but the magnitude of increased acidityt is not enough to induce the mass apoptosis of cancer cells below this temperature. Not yet researches have found that there is a progressive causal relationship between the tumor's hyperthermia therapy range of 43° C.-44° C., the increase of intratumoral acidity to pH<5, and the mass apoptosis of cancer cells.

The advantageous effects of the present disclosure:

In the present disclosure, the overall acidity of tumor tissue is enhanced through physical or biochemical methods to release out a signal of serious shortage of logistics supply inside the cancer cells, can induce cancer cells to spontaneously restart the apoptosis program, then stop protein synthesis and DNA replication of cancer cells, and reduce the consumption of proliferation through apoptosis, which is conducive to the survival and self-protection of cancer cells. At this time, although the cancer cells are not completely dead, they are already in a dormant and half-dead state, and no longer differentiate and proliferate, actuall already lose the function of differentiation and proliferation, and stop metastasis and diffusion, achieving the purpose of curing tumors. In the present disclosure, inducing the mass apoptosis of cancer cells by enhancing the overall acidity of the tumor interior not only realizes to kill cancer cells, but also avoids damage to normal cells, and is a safe and efficient method for tumor treatment.

### DETAILED DESCRIPTION OF THE EMBODIMENTS

The preferred embodiments of the present disclosure are described in detail below so that the advantages and features of the present disclosure may be more easily understood by those skilled in the art so as to make a clearer and clearer definition of the protection scope of the present disclosure.

A method for inducing mass apoptosis of cancer cells by enhancing overall acidity in tumor, wherein the method comprises steps of reducing overall pH in tumor tissue by adopting a physical method or a biochemical method so as to enhance overall acidity in tumor tissue, which in turn induce mass apoptosis of cancer cells.

The physical or biochemical methods include a variety of methods, such as by injecting an acid liquid directly into the tumor tissue or by indirectly promoting the overall acidification in tumor tissue, any methods as long as that can reduce the overall pH value of the tumor tissue are all feasible.

In one embodiment, the key step of reducing the overall pH in tumor tissue includes making the pH of extracellular micro-environment of cancer cells in tumor lower than 5.

In one embodiment, base on the same direction synergic interaction between the changes of intracellular acidity and extracellular acidity in tumor (pH<6.5), reducing overall pH in tumor tissue may be performed by injecting an acid directly into tumor tissue or by aggravating anaerobic respiration of cancer cells through hyperthermia therapy to indirectly promote the overall acidification in tumor tissue.

In one embodiment, for a single tumor without metastasis and diffusion, a fashion of injecting an acid directly into tumor tissue must make the pH of extracellular micro-environment of cancer cells to be lower than 5.

In one embodiment, for a single tumor without metastasis and diffusion, a fashion of injecting a liquid of an acid substance directly into tumor tissue must make the pH of extracellular micro-environment of cancer cells to be lower than 5, thus inducing mass apoptosis of cancer cells. the acid substance described above may be any one of lactic acid, acetic acid and hydrochloric acid, etc.

In one embodiment, for promoting indirectly the tumor tissue to overall acidification, a method of hyperthermia therapy to the whole body or hyperthermia therapy to tumor tissue region can be applied to aggravate the anaerobic respiration of cancer cells, and promote the acidification of intracellularly tissues and extracellular tissues of cancer cells, thus enhancing the overall acidity in tumor, when the pH of extracellular micro-environment of cancer cells is lower than 5, mass apoptosis of cancer cells can be sure induced.

In one embodiment, for a single tumor without metastasis and diffusion, a hyperthermia therapy can also be applied to tumor tissue region, so to aggravate the anaerobic respiration of cancer cells and promote the acidification of intracellularly tissues and extracellular tissues of cancer cells, thus enhance the overall acidity inside the tumor and induce apoptosis of cancer cells. The higher the acidity in the tumor is, the higher the ratio of apoptosis of cancer cells. Only when the pH value of extracellular micro-environment of cancer cells is lower than 6.2, the apoptosis process can be initiated. Only when the pH value of extracellular micro-environment of cancer cells is reduced lower than 5.0, the mass apoptosis of cancer cells can be induced.

In one embodiment, for metastatic and diffuse tumors, a hyperthermia therapy can be applied to whole body of the patient to aggravate the anaerobic respiration of cancer cells and promote the acidification of intracellularly tissues and extracellular tissues of cancer cells, thus fully enhancing the overall acidity in tumor, when the pH of extracellular micro-environment of cancer cells is lower than 5, the mass apoptosis of cancer cells can be induced.

In one embodiment, when a hyperthermia therapy is used, the inside of tumor tissue is heated to a temperature of 43° C.-44° C., which is more conducive to promote the acidity to enhance quickly in the tumor, inducing a highest rate of apoptosis of cancer cells, so as to achieve the mass apoptosis of cancer cells.

In one embodiment, during the hyperthermia therapy, the optimal temperature range for the hyperthermia therapy is 43° C.-44° C., the hyperthermia therapy is carried out once a day, with an accumulated time for being not less than 30 minutes each hyperthermia therapy. in order to strengthen the effect, the hyperthermia therapy is repeated once the next day

The overall acidification of the tumor tissue releases a signal that the cancer cells are suffering from a severe shortage of supplies, it indicates that cancer cells can gain self-protection only when they initiate the apoptotic program to reduce the huge consumption for oxygen and nutrients. Although the apoptotic cancer cells are not thoroughly death, Protein synthesis and DNA replication of cancer cells have been inhibited through apoptosis. At this time, the cancer cells lose the function of differentiation and proliferation, and difficult to transfer and spread, it's the equivalent of castration, In effect it's equal to the cance being cured.

### EXAMPLE 1

For a single tumor without metastasis and diffusion, to injecte directly acid liquid into tumor tissue may enhance the overall acidity in the tumor, thus induce mass apoptosis of cancer cells in the tumor, In the end the tumors can be cured.

The patient Li, female, 39 years old. In a health examination 2 years ago, a 3.2*2.5*1.8 cm tumor with rough edges and a little hard feeling by hand was found in her right breast by mammary gland molybdenum target device. The patient was diagnosed with invasive ductal carcinoma of the breast by puncture biopsy; Positron emission tomography-computed tomography (PET-CT) showed a single tumor, the imaging showed obviously radioactive concentration, there were not other abnormal dense shadows and swollen lymph node. The diagnosis was a single tumor without metastasis and diffusion.

Treatment process: Medical acetic acid was dissolved in sterile water for injection, and 50,000 units of penicillin sodium were added to make 20 ml of a weak acid with a pH of 2. The drainage needle was preseted at the inner edge of the tumor adventitia being used to detect the acidity of the tumor, and the infusion needle was punctured to the center of her tumor, and the weak acid solution no less than 25% of the tumor volume was slowly injected into the tumor in not less than 50 minutes through the infusion needle with the aid of an injection pump. During the injection, by controlling the speed of the injection pump, the pH of the tumor was constantly kept at 4-5, with the maximum being not more than 5. The treatment was performed once every two days, usually acid injection is done 2-3 times. Then reviewed once every half month with mammary gland molybdenum target. The first three reexaminations showed that there was no change in tumor growth. After the fourth reexaminations, the tumor began to atrophy slowly. After 3 months, hand felt original tumor had become soft. After 6 months, the PET-CT of the tumor showed a spotted streak shadow, and the spotted streak shadow area was about 40% smaller than before treatment, the radioactive concentration in that area was significantly weakened and was close to normal. One year later, no active cancer cells were detected by the biopsy.

### EXAMPLE 2

For early-stage tumors without obvious metastasis and diffusion, the hyperthermia therapy is used to tumor tissue region with precise radiation in order to aggravate the anaerobic respiration of the cancer cells, to promote the acidification of intracellularly tissues and extracellular tissues of cancer cells through the accumulation of lactic acid, thus enhance the overall acidity inside the tumor and induce apoptosis of cancer cells.

Patient Liu, male, 43 years old, a black nevus about the size of a soybean was found on the left hand fifteen years ago, and the nevus grew slowly at first. Two years ago, the original nevus gradually enlarged and the pigmentation deepened. As the enlargement, the lesions area appeared nodular, and the ulceration and bleeding on the surface were difficult to heal. There were irregular pigmented halos around, but no swelling of the axillary lymph nodes was found. The diagnosis confirmed a malignant melanoma.

Treatment process: The whole left hand and left arm were sterilized, lidocaine was injected from the shoulder to the left arm for local anesthesia, and then the whole left arm and left hand were wrapped with the sterilized film, then place the entire left arm and left hand in a thermostatic water tank with the temperature of 44-45° C. for water bath. The first water bath lasted for 40 minutes, and the next day a hyperthermia therapy was repeated for reinforcement, with the hyperthermia therapy time of 35 minutes. One week later, the ulcer began to heal and scab. One month later, the scab fell off, subcutaneous mass began to soften and atrophy. One year later, the mass disappeared, light black scar formed at the nevus, and no active cancer cells were found in the puncture biopsy of the lesion.

### EXAMPLE 3

For metastatic and diffuse middle-stage tumors, microbial infections were used to induce high fevers to aggravate the anaerobic respiration of the cancer cells, through the accumulation of lactic acid to promote the acidification of intracellularly tissues and extracellular tissues of cancer cells, enhance the overall acidity inside the tumor, thus induce mass apoptosis of cancer cells in the tumors, last the tumor can be cured.

The patient Zhu, male, 81 years old, was hospitalized 6 years ago due to repeated low-grade fever. At that time, a huge lump with unclear tumor borders in the upper lobe of the right lung was found by PET-CT, CT value was 40 Hu, the size was 7.3*5.5*3.8 cm, The imaging of PET showed radioactive concentration, SUV value was 18.6. The tubercle shadow lay behind the mediastinal internal vena cava, the local density was high, CT value of 48 Hu. The imaging of PET showed radioactive concentration, SUV value was 4.3, suggesting lymph node metastasis. Healing process: The result of puncture biopsy was diagnosed as mid-stage squamous cancer of the upper right lung. During hospitalization, accidental infection caused a high fever due to infusion, the body temperature was over 42° C. The fever subsided after about 40 minutes of rescue. Afterwards, due to the high age of the patient, it was not suitable for surgical therapy, radiotherapy and chemotherapy, therefore the patient gave up conventional treatment. The results was that after accidental high fever his tumor was cured without other treatment. A special emphasis on that the use of any anti-cancer drugs during his whole treatment was completely excluded. He has been symptom-free for more than 6 years. He was 81 years old at that time and very healthy. Recent PET-CT check showed: the original lump in the upper lobe of the right lung disappeared and only multiple patches were seen. The result of PET showed that the lesion region had no abnormal increase in radioactivity uptake.

### EXAMPLE 4

For metastatic and diffuse advanced breast cancer, a multi-source antenna array microwave hyperthermia therapy device was used to radiate tumor tissue region with wide-area precise hyperthermia, then to promote tumor tissue acidification, so as to induce mass apoptosis of these cancer cells. Compared with the tumor without experiment of hyperthermia therapy and acidification, verified the method of enhancing the overall acidity in the tumor to induce mass apoptosis of cancer cell has a reliable effect.

The patient Xu, female, 56 years old. One year ago, two lumps of different sizes were showed in the left breast by molybdenum target mammography of breast: one lump(A) was 2.2*2*1.3 cm, and the other lump (B) was 1.5*1*0.6 cm. and the lump(C) of right breast was 2.5*2*1.8 cm in size. These three lumps had rough edges and a hand feels a little hard. There was discharge in the left nipple. It was diagnosed as invasive ductal carcinoma of the breast by puncture biopsy. PET-CT showed that multiple lumps were obviously radioactive concentration, The patient presented with lung metastasis and multiple bone metastases, and the axillary lymph nodes on both sides were obviously enlarged. The patient was diagnosed as advanced breast cancer.

Treatment process: In view of the occurrence of distant metastasis, the patient gave up surgical therapy and only received chemotherapy, local acidification and hyperthermia therapy for palliative conservative treatment. First, a course of CMF(cyclophosphamide, methotrexate and fluorouracil) regimen chemotherapy control was performed, and then the lump(A) in left breast was injected with acid for 2 times (the method was the same as in Example 1), and using the precise optical fiber temperature measurement-control technology, the right breast was irradiated by wide-area hyperthermia therapy with a multi-source antenna array microwave hyperthermia devic. Precision control temperatureis carried out with the cooperation of optical fiber temperature measurement technology during hyperthermia therapy (fiber-optic temperature measuring device was placed in the right breast through the incision). Considering that the maximum depth of microwave irradiation was only 6cm, the right breast was squashed by a plastic sheet during irradiation, it was conducive that the lump(C) of right breast was in the range of the penetration depth of the hyperthermia therapy device, and the temperature of hyperthermia therapy was controlled within the range of 43-44° C., the duration of the hyperthermia therapy was 40 minutes. Three months later, the PET-CT showed that the acid-infused the lump(A) in the left breast did not increase and slightly atrophied, and its radioactive concentration was significantly reduced; The symptoms of the lump(B) without acid injection and hyperthermia therapy did not improve, but slightly enlarged, The radioactive concentration of the lump did not reduce;The lump(C) of right breast did not enlarge and slightly shrank after the hyperthermia therapy, and its radioactive concentration was significantly reduced;The other lung metastases and multiple bone metastases were not affected by the hyperthermia therapy and acid injection, and were still in the progressive stage, all varying degrees of increase, the radioactive concentration was not improved. The patient died 9 months later before death, the acid-injected lump(A) and hyperthermia treated lump(C) did not worsen, and there was further atrophy. The lump(B) that was not subject to hyperthermia therapy and acid-injection deteriorated significantly.

In the present disclosure, for reducing the overall pH of the tumor tissue, making the overall acidity of the intracellular tissues and extracellular tissues of the cancer cells in the tumor is enhanced by adjusting the overall acid-base property in the tumor, thereby promoting the mass apoptosis of the cancer cells, in effect it's equal to the cancer being cured.

In the present disclosure, promotion of the mass apoptosis of cancer cells by enhancing the overall acidity of tumor tissues, it only requires that the pH of the extracellular micro-environment of cancer cells in the tumor should be lower than 5, and the stronger the acidity of tumor cells is, the higher the apoptosis rate of cancer cells is.

In the present disclosure, there are included a variety of methods to enhance the overall acidity in the tumor. The method of directly injecting an acid into the tumor tissue or thermal therapy measures such as physical diathermy, microbial infection and hyperthermia therapy can be all used, as long as it can enhance the overall acidity of the tumor tissue while not too enhancing the acidity of normal cells are all feasible.

In the present disclosure, for a single tumor without metastasis and diffusion, the method of directly injecting an acidic liquid into the tumor tissue or radiating tumor tissue region with wide-area hyperthermia therapy may be adopted, only the pH of the extracellular micro-environment of the cancer cells in the tumor is lower than 5 can induce mass apoptosis of cancer cells without damaging normal cells, can achiev the purpose of killing cancer cells.

In the present disclosure, for metastatic and diffuse tumors, the anaerobic respiration of all tumor tissues can be indirectly induced through hyperthermia therapy, the form of hyperthermia therapy can be physical diathermy, microbial infection and fever, etc, as long as it can enhance the overall acidity of the tumor tissue while not too enhancing the acidity of normal cells are all feasible. Only when the pH of the extracellular micro-environment of cancer cells in the tumor is lower than 5, can the mass apoptosis of cancer cells be induced. At the same time, the temperature should be controlled to ensure that normal cells are not too acidified and are not damaged.

The hyperthermia therapy to metastatic tumors must be hyperthermia therapy to the whole body, so that full coverage of all cancer cells is ensured. The temperature range for hyperthermia therapy is 43° C.-44° C., and the hyperthermia therapy is carried out once a day, with the accumulated duration of time for each hyperthermia therapy being not less than 30 minutes. in order to intensive effect the hyperthermia therapy is repeated once the next day.

The disclosure discloses a method for inducing mass apoptosis of cancer cell by enhancing the overall acidity in the tumor. The method reduces the overall pH of tumor tissue by adjusting the acidity and basicity of the tumors, so as to enhance apoptosis of cancer cell. In order to induce apoptosis of cancer cells, the pH of the extracellular micro-environment of cancer cells must be lower than 6.2. The stronger the acidity in the tumor is, the higher the apoptosis rate of cancer cells is. When the pH of the extracellular micro-environment of cancer cells in the tumor is lower than 5, the mass apoptosis of cancer cells can be induced. There are a variety of measures to enhance the acidity of the internal environment of tumor, the method of directly injecting an acid into the tumor tissue or hyperthermia therapy such as physical diathermy, microbial infection and fever may be all used, as long as it can enhance the overall acidity of the tumor while not too enhancing the acidity of normal cells. In the present disclosure, enhance of the overall acidity in the tumor to induce the mass apoptosis of cancer cells not only can realize the curing of cancer cells, but also can avoid damaging to normal cells, and is a safe and efficient method for tumor treatment.

In the present disclosure, the principle and the mechanism of treating tumor by induction of the mass apoptosis of cancer cells through the overall acidification of the tumor are simple and a scientific the technology is easy to realize and the harm to normal cells is very small. The method in the present disclosure has good effect and high safety, and has great popularization and application value.

For those skilled in the art, it is obvious that the present disclosure is not limited to the details of the embodiments, and the present disclosure can be implemented in other specific forms without departing from the spirit or basic characteristics of the present disclosure. Therefore, from any point of view, the embodiments should be regarded as exemplary and non-limiting. The scope of the present disclosure is limited by the appended claims rather than the above description. Therefore, it is intended to include all changes within the meaning and scope of the equivalent elements of the claims in the present invention. Any reference signs in the claims shall not be regarded as limiting the claims concerned.

In addition, it should be understood that although the description is described according to the embodiments, it is not that every embodiment includes only an independent technical solution. This description above is only for purpose of clarity. Those skilled in the art should consider the specification as a whole, and the technical solutions in the embodiments can also be appropriately combined to form other embodiments that can be understood by those skilled in the art.

1. A method for inducing mass apoptosis of cancer cells by enhancing overall acidity in tumor, comprising reducing overall pH in tumor tissue by adopting a physical or biochemical method, so as to enhance overall acidity in tumor tissue and induce mass apoptosis of cancer cells.

2. The method according to claim 1, wherein reducing overall pH in tumor tissue comprises needing to make the pH of extracellular micro-environment of cancer cells in tumor lower than 5, in order to induce mass apoptosis of cancer cells.

3. The method according to claim 1, wherein base on the same direction synergic interaction between the changes of intracellular acidity and extracellular acidity in tumor, reducing overall pH in tumor tissue can be performed by injecting an acid directly into tumor tissue or by hyperthermia therapy aggravateing anaerobic respiration of cancer cells in order to indirectly induce the overall acidification in tumor tissue.

4. The method according to claim 3, wherein injecting an acid directly into tumor tissue comprises an acid liquid is injected directly into tumor tissue to promote the pH of extracellular micro-environment of cancer cells to be lower than 5, thus inducing mass apoptosis of cancer cells.

5. The method according to claim 3, wherein the hyperthermia therapy comprises aggravating the anaerobic respiration of cancer cells through hyperthermia therapy to a whole body or hyperthermia therapy to tumor tissue region, so as to promote the acidification of intracellularly tissues and extracellular tissues of cancer cells, intent is to promote the pH of extracellular micro-environment of cancer cells to be lower than 5, thus fully enhancing the overall acidity in tumor, in the end inducing mass apoptosis of cancer cells.

6. The method according to claim 5, wherein internal temperature of tumor tissue is 43° C.-44° C. during the hyperthermia therapy.

7. The method according to claim 6, wherein the optimal temperature range for the hyperthermia therapy is 43° C.-44° C., the hyperthermia therapy is carried out once a day, with accumulated time for each hyperthermia therapy being not less than 30 minutes, in order to intensive effect the hyperthermia therapy is repeated once the next day.
